SEQUENCE LISTING (14 sequences)

SEQ ID NO: 1 - protein, 11 aa, Artificial Sequence
  Peptide based on human Netrin-1 sequence
  MISC_FEATURE (1)..(1): Xaa is Ala, Asn, Cys, Ser, or Thr, and wherein when X1 is Cys, it is linked to either the cysteine residue at the fourth amino acid position via a disulfide bond or an ethylene oxide
  MISC_FEATURE (2)..(2): Xaa is present or absent, and if present, Xaa is Ala, Asp, Ile, Leu, Met, Phe, Pro, Trp, or Val, and wherein this Xaa is present where the Xaa in the third amino acid position is absent
  MISC_FEATURE (3)..(3): Xaa is present or absent, and if present, Xaa is Asn, Arg, Asp, Cys, Gln, Glu, Gly, Ser, Thr, or Tyr, and wherein this Xaa is present where the Xaa in the second amino acid position is absent
  MISC_FEATURE (5)..(5): Xaa is Arg, His, or Lys
  MISC_FEATURE (6)..(6): Xaa is Arg, Asp, Glu, His, Lys, Phe, Trp, or Tyr
  MISC_FEATURE (7)..(7): Xaa is Asn, Cys, Gln, Gly, Ser, Thr, Tyr, or Val
  MISC_FEATURE (8)..(8): Xaa is present or absent, and if present, Xaa is Asn, Gly, His, Ile, Thr, or Val
  MISC_FEATURE (10)..(10): Xaa is present or absent, and if present, Xaa is Ala, Asn, Ile, Leu, Met, Phe, Pro, Thr, Trp, or Val
XXXCXXXXTXG

SEQ ID NO: 2 - protein, 9 aa, Artificial Sequence
  Peptide based on human Netrin-1 sequence
  MISC_FEATURE (1)..(1): Wherein diethylene glycol (mini-PEG) is linked thereto
CDCRHNTAG

SEQ ID NO: 3 - protein, 10 aa, Artificial Sequence
  Peptide based on human Netrin-1 sequence
  MISC_FEATURE (1)..(1): Wherein diethylene glycol (mini-PEG) is linked thereto
CLNCRHNTAG

SEQ ID NO: 4 - protein, 12 aa, Artificial Sequence
  Peptide based on human Netrin-1 sequence
  MISC_FEATURE (1)..(1): Wherein diethylene glycol (mini-PEG) is linked thereto
CPCKDGVTIGIT

SEQ ID NO: 5 - protein, 9 aa, Artificial Sequence
  Peptide based on human Netrin-1 sequence
SDCRHNTAG

SEQ ID NO: 6 - protein, 9 aa, Artificial Sequence
  Peptide based on human Netrin-1 sequence
TDCRHNTAG

SEQ ID NO: 7 - protein, 9 aa, Artificial Sequence
  Peptide based on human Netrin-1 sequence
  DISULFID (1)..(3)
CDCRHNTAG

SEQ ID NO: 8 - protein, 54 aa, Artificial Sequence
  Peptide based on human Netrin-1 sequence
CKCNGHAARCVRDRDDSLVCDCRHNTAGPECDRCKPFHYDRPWQRATAREANEC

SEQ ID NO: 9 - protein, 61 aa, Artificial Sequence
  Peptide based on human Netrin-1 sequence
CNCNLHARRCRFNMELYKLSGRKSGGVCLNCRHNTAGRHCHYCKEGYYRDMGKPITHRKAC

SEQ ID NO: 10 - protein, 48 aa, Artificial Sequence
  Peptide based on human Netrin-1 sequence
CDCHPVGAAGKTCNQTTGQCPCKDGVTGITCNRCAKGYQQSRSPIAPC

SEQ ID NO: 11 - protein, 9 aa, Artificial Sequence
  Peptide based on human Netrin-1 sequence
CDCRHNTAG

SEQ ID NO: 12 - protein, 10 aa, Artificial Sequence
  Peptide based on human Netrin-1 sequence
CLNCRHNTAG

SEQ ID NO: 13 - protein, 11 aa, Artificial Sequence
  Peptide based on human Netrin-1 sequence
CPCKDGVTGIT

SEQ ID NO: 14 - protein, 16 aa, Artificial Sequence
  Peptide based on human Netrin-1 sequence
HPVGAAGKTCNQTTGQ

(19) United States
(12) Patent Application Publication
(10) Pub. No.: US 2021/0371463 A1
(43) Pub. Date: Dec. 2, 2021

(54) Modified Netrin-1 Peptides and Compositions for Cardioprotection

(71) Applicant: The Regents of the University of California, Oakland, CA (US)

(72) Inventor: Hua Cai, Los Angeles, CA (US)

(21) Appl. No.: 17/286,851

(22) PCT Filed: Dec. 11, 2019
(86) PCT No.: PCT/US2019/065587

§ 371 Date: Apr. 20, 2021

Related U.S. Application Data
(60) Provisional application No. 62/778,406, filed on Dec. 12, 2018.

(51) Int. Cl. C07K 7/06

(52) U.S. Cl. CPC C07K 7/06; A61K 38/00

(57) ABSTRACT

Disclosed herein are modified netrin-1 peptides and compositions thereof and methods of using thereof to confer cardioprotection. In some embodiments, the present invention provides methods of using one or more modified netrin-1 peptides and compositions thereof In some embodiments, the present invention provides a method of stimulating, increasing, or enhancing nitric oxide production by endothelial cells, which comprises administering to the endothelial cells one or more modified netrin-1 peptides or a composition thereof.

### CROSS-REFERENCE TO RELATED APPLICATIONS

This application claims the benefit of U.S. Patent Application No. 62/778,406 filed Dec. 12, 2018, which is herein incorporated by reference in its entirety.

### ACKNOWLEDGEMENT OF GOVERNMENT SUPPORT

This invention was made with Government support under Grant Number HL119968, awarded by the National Institutes of Health. The Government has certain rights in the invention.

### REFERENCE TO A SEQUENCE LISTING SUBMITTED VIA EFS-WEB

The content of the ASCII text file of the sequence listing named “20181210 034044 194P1 seq ST25” which is 6.12 kb in size was created on Dec. 11, 2018, and electronically submitted via EFS-Web herewith the application is incorporated herein by reference in its entirety.

### BACKGROUND OF THE INVENTION

### 1. Field of the Invention

The present invention generally relates to modified netrin-1 peptides and compositions thereof and methods of using thereof.

### 2. Description of the Related Art

Netrins and their receptors are well known in the art, as exemplified in U.S. Pat. Nos. 5,565,331; 6,096,866; 6,017,714; 6,309,638; 6,670,451; and 8,168,593; and in US20060019896 and US20060025335.

Netrin-1 is a secreted molecule that is largely known to play a defined role in guiding vertebrate commissural axons in neuronal development. See Kennedy et al. (1994) Cell 78:425-35; Serafini et al. (1994) Cell 78:409-24; and Serafini et al. (1996) Cell 87:1001-14. Recent studies have further demonstrated a critical role of netrin-1 in endothelial cell proliferation, migration, and angiogenic signaling, in addition to morphogenesis of epithelial cells. See Park et al. (2004) PNAS USA 101:16210-5; Carmeliet et al. (2005) Nature 436:193-200; Nguyen et al. (2006) PNAS USA 103:6530-5; Wilson et al. (2006) Science 313:640-4; Liu et al. (2004) Curr Biol 14:897-905. At least eight netrin receptors have been characterized in neurons, vascular system, and other cell types in mammals. These include deleted in colorectal cancer (DCC), UNCSA, B, C, D, neogenin, α6β4, and α3β1 integrins. See Tessier-Lavigne et al. (1996) Science 274:1123-33; Huber et al. (2003) Annu Rev Neurosci 26:509-63; Cirulli et al. (2007) Nat Rev Mol Cell Biol 8:296-306; and Yebra et al. (2003) Dev Cell 5:695-707. Netrin-1 binding to DCC mediates attractive outgrowth of axons, as well as positive angiogenic signaling in endothelial cells. In contrast, the UNCSB receptor appears repulsive, mediating cellular effects such as filopodial retraction, particularly in developing capillaries. See Lu et al. (2004) Nature 432:179-86; and Larrivee et al. (2007) Genes Dev 21:2433-47.

### SUMMARY OF THE INVENTION

In some embodiments, the present invention provides modified netrin-1 peptides as described herein and compositions thereof.

In some embodiments, the present invention provides methods of using one or more modified netrin-1 peptides and compositions thereof. In some embodiments, the present invention provides a method of stimulating, increasing, or enhancing nitric oxide production by endothelial cells, which comprises administering to the endothelial cells one or more modified netrin-1 peptides or a composition thereof. In some embodiments, the present invention provides a method of stimulating or inducing phosphorylation of ERK1/2 and/or eNOS in endothelial cells, which comprises administering to the endothelial cells one or more modified netrin-1 peptides or a composition thereof In some embodiments, the present invention provides a method of treating, inhibiting, or reducing an injury to a tissue or organ having endothelial cells which comprises stimulating, increasing, or enhancing nitric oxide production by the endothelial cells and/or stimulating or inducing phosphorylation of ERK1/2, eNOS, or both in the endothelial cells by administering to the endothelial cells, before, during, and/or after the injury, one or more modified netrin-1 peptides or a composition thereof. In some embodiments, the endothelial cells are vascular endothelial cells. In some methods according to the present invention, the administration to the endothelial cells is in vivo administration. In some embodiments, the injury is caused by superoxide production, ischemia/reperfusion, or myocardial infarction. In some embodiments, the tissue is cardiac tissue. In some embodiments, the organ is a heart. In some embodiments, the injury is caused by myocardial infarction and the administration reduces the infarct size of the heart. In some embodiments, the present invention is directed to treating or inhibiting restenosis in a subject which comprises administering to the subject a therapeutically effective amount of one or more one or more modified netrin-1 peptides, before, during, or after an injury to endothelial cells in a blood vessel. In some embodiments, the present invention provides a method of treating, inhibiting, or reducing an ischemia/reperfusion injury to an organ, e.g., a heart, in a subject, comprising administering a therapeutically effective amount of one or more modified netrin-1 peptides or a composition thereof to the subject, thereby treating, inhibiting, or reducing the ischemia/reperfusion injury. In some embodiments, the present invention provides a method of decreasing or reducing the infarct size of a heart in a subject resulting from an ischemia/reperfusion injury, comprising administering a therapeutically effective amount of one or more modified netrin-1 peptides or a composition thereof to the subject, thereby decreasing or reducing the infarct size. In some embodiments, the subject is a mammalian subject. In some embodiments, the subject is an animal model, e.g., a mouse. In some embodiments, the subject is a human. In some embodiments, the subject being treated with one or more modified netrin-1 peptides or compositions according to the present invention is one who is in need thereof. Subjects who are in need thereof include those who may benefit from stimulating, increasing, or enhancing nitric oxide production, those who may benefit from stimulating or inducing phosphorylation of ERK1/2 and/or eNOS, those who have or may have a tissue or organ injury resulting from increased production of reactive oxygen species or oxidative stress, ischemia/reperfusion, or myocardial infarction, and those who will be or will likely be exposed to increased superoxide production, ischemia/reperfusion conditions, or myocardial infarction.

In some embodiments, the present invention provides a human-made package, e.g., a kit, comprising therein one or more modified netrin-1 peptides or a composition thereof. In some embodiments, the human-made package further includes a drug delivery device. In some embodiments, the present invention provides a device comprising therein one or more modified netrin-1 peptides or a composition thereof.

Both the foregoing general description and the following detailed description are exemplary and explanatory only and are intended to provide further explanation of the invention as claimed. Any accompanying drawings are included to provide a further understanding of the invention and are incorporated in and constitute part of this specification, illustrate several embodiments of the invention, and together with the description serve to explain the principles of the invention.

### DETAILED DESCRIPTION OF THE INVENTION

Previously, netrin-1 and netrin-1 peptide fragments were found to exhibit cardioprotective activity when administered to subjects. See PCT/US2011/038277, PCT/US2015/023248, and Li & Cai (2015) Am J Physiol Cell Physiol 309:C100-106, which are herein incorporated by reference in their entirety. The prior art netrin-1 peptide fragments are referred to herein as “prior art peptides”. When delivered to the heart, the prior art peptides activate the protective pathway turned on by netrin-1, namely DCC-dependent activation of ERK1/2 and eNOS. ERK1/2, and eNOSs₁₁₇₇ (1177 residue for human/mouse while 1179 for bovine) phosphorylation were time-dependently increased by the prior art peptides in cultured endothelial cells, which is believed to increase nitric oxide (NO) production to exert cardioprotection.

As disclosed herein, the instant inventors further modified the prior art peptides and assayed their cardioprotective efficacy against ischemia reperfusion (I/R) injuries. Analysis of post-FR infarct size shows a significant reduction in myocardial injury after treatment with the modified netrin-1 peptides. In fact, the modified netrin-1 peptides provide a significantly greater reduction of infarct size—about a 14-40% greater reduction of infarct size—compared to the prior art peptides.

Therefore, the present invention provides modified netrin-1 peptides and compositions and methods thereof. As used herein, a “modified netrin-1 peptide” refers to a peptide or protein that comprises, consists essentially of, or consists of SEQ ID NO: 1 as follows:

(SEQ ID NO: 1)
X1-X2-X3-C-X4-X5-X6-X7-T-X8-G

wherein

X1 is Ala, Asn, Cys, Ser, or Thr, preferably X1 is Cys, Ser, or Thr, wherein when X1 is Cys, it is linked, e.g., attached, covalently or non-covalently, to either the cysteine residue at the fourth amino acid position via a disulfide bond or an ethylene oxide compound;

X2 is present or absent, and if present, X2 is Ala, Asp, Ile, Leu, Met, Phe, Pro, Trp, or Val, preferably X2 is Leu or Pro;

X3 is present or absent, and if present, X3 is Asn, Arg, Asp, Cys, Gln, Glu, Gly, Ser, Thr, or Tyr, preferably X3 is Asn or Asp;

X4 is Arg, His, or Lys, preferably X4 is Arg or Lys;

X5 is Arg, Asp, Glu, His, Lys, Phe, Trp, or Tyr, preferably X5 is Asn, Asp, or His;

X6 is Asn, Cys, Gln, Gly, Ser, Thr, Tyr, or Val, preferably X6 is Asn or Gly;

X7 is present or absent, and if present, X7 is Asn, Gly, His, Ile, Thr, or Val, preferably X7 is Val; and

X8 is present or absent, and if present, X8 is Ala, Asn, Ile, Leu, Met, Phe, Pro, Thr, Trp, or Val, preferably X8 is Ala; and

wherein X2, X3, or both X2 and X3 are present.

In some embodiments, the ethylene oxide compound is polyethylene glycol (PEG), polyethylene oxide (PEO), and polyoxyethylene (POE), methoxypolyethylene glycol (MPEG), or monomethoxypolyethylene glycol (mPEG), or diethylene glycol (mini-PEG), preferably the ethylene oxide compound is mini-PEG.

In some embodiments, the modified netrin-1 peptides are about 8-60, about 8-55, about 8-50, about 8-45, about 8-40, about 8-35, about 8-30, about 8-25, about 8-20, about 8-15, about 8-12, 8-11, about 9-60, about 9-55, about 9-50, about 9-45, about 9-40, about 9-35, about 9-30, about 9-25, about 9-20, about 9-15, about 9-12, or 9-11 amino acid residues long. In some embodiments, the modified netrin-1 peptides are 8, 9, 10, or 11 amino acid residues long.

As used herein, a peptide that “comprises” a given sequence means that the peptide may include additional amino acid residues, amino acid isomers, and/or amino acid analogs at the N-terminus, the C-terminus, or both. The additional residues may or may not change the activity or function of the given sequence, i.e., the peptide having the additional residues, isomers, or analogs may have a different activity or function as compared to the given sequence itself (without the additional residues, isomers, or analogs). As used herein, a peptide that “consists essentially of” a given sequence means that the peptide may include additional amino acid residues, amino acid isomers, and/or amino acid analogs at the N-terminus, the C-terminus, or both, so long as they do not materially change the function or activity of the given sequence, i.e., the peptide having the additional residues, isomers, or analogs has an activity and function that are substantially similar to that of the given sequence itself. As used herein, a peptide that “consists of” a given sequence means that the peptide does not include additional amino acid residues, amino acid isomers, and/or amino acid analogs at either the N-terminus or the C-terminus.

In some embodiments, modified netrin-1 peptides may be isolated. As used herein, an “isolated” compound refers to a compound which is isolated from its native environment. For example, an isolated peptide is one which does not have its native amino acids, which correspond to the full-length polypeptide, flanking the N-terminus, C-terminus, or both. For example, an isolated V1-9aa peptide refers to a peptide having amino acid residues (304-312 aa) of V1, which may have non-native amino acids at its N-terminus, C-terminus, or both, but does not have a proline amino acid residue following its 9^{th} amino acid residue at the C-terminus, or a valine amino acid residue immediately preceding the cysteine amino acid residue at its N-terminus, or both. As another example, an isolated peptide can be one which is immobilized to a substrate with which the peptide is not naturally associated. As a further example, an isolated peptide can be one which is linked to another molecule, e.g., a PEG compound, e.g., mPEG, with which the peptide is not naturally associated.

In some embodiments, modified netrin-1 peptides may comprise one or more natural amino acids, unnatural amino acids, or a combination thereof. The amino acid residues of the peptide may be D-isomers, L-isomers, or both. The peptide may be composed of α-amino acids, β-amino acids, natural amino acids, non-natural amino acids, amino acid analogs, or a combination thereof. Amino acid analogs include β-amino acids and amino acids where the amino or carboxy group is substituted by a similarly reactive group (e.g., substitution of the primary amine with a secondary or tertiary amine, or substitution of the carboxy group with an ester).

Examples of β-amino acid analogs include cyclic β-amino acid analogs; β-alanine; I-β-phenylalanine; I-1,2,3,4-tetrahydro-isoquinoline-3-acetic acid; I-3-amino-4-(1-naphthyl)-butyric acid; I-β-amino-4-(2,4-dichlorophenyl)butyric acid; I-3-amino-4-(2-chlorophenyl)-butyric acid; I-3-amino-4-(2-cyanophenyl)-butyric acid; I-3-amino-4-(2-fluorophenyl)-butyric acid; I-3-amino-4-(2-furyl)-butyric acid; I-3-amino-4-(2-methylphenyl)-butyric acid; I-3-amino-4-(2-naphthyl)-butyric acid; I-3-amino-4-(2-thienyl)-butyric acid; I-3-amino-4-(2-trifluoromethylphenyl)-butyric acid; I-3-amino-4-(3,4-dichlorophenyl)butyric acid; I-3-amino-4-(3,4-difluorophenyl)butyric acid; I-3-amino-4-(3-benzothienyl)-butyric acid; I-3-amino-4-(3-chlorophenyl)-butyric acid; I-3-amino-4-(3-cyanophenyl)-butyric acid; I-3-amino-4-(3-fluorophenyl)-butyric acid; I-3-amino-4-(3-methylphenyl)-butyric acid; I-3-amino-4-(3-pyridyl)-butyric acid; I-3-amino-4-(3-thienyl)-butyric acid; I-3-amino-4-(3-trifluoromethylphenyl)-butyric acid; I-3-amino-4-(4-bromophenyl)-butyric acid; I-3-amino-4-(4-chlorophenyl)-butyric acid; I-3-amino-4-(4-cyanophenyl)-butyric acid; I-3-amino-4-(4-fluorophenyl)-butyric acid; I-3-amino-4-(4-iodophenyl)-butyric acid; I-3-amino-4-(4-methylphenyl)-butyric acid; I-3-amino-4-(4-nitrophenyl)-butyric acid; I-3-amino-4-(4-pyridyl)-butyric acid; I-3-amino-4-(4-trifluoromethylphenyl)-butyric acid; I-3-amino-4-pentafluoro-phenylbutyric acid; I-3-amino-5-hexenoic acid; I-3-amino-5-hexynoic acid; I-3-amino-5-phenylpentanoic acid; I-3-amino-6-phenyl-5-hexenoic acid; (S)-1,2,3,4-tetrahydro-isoquinoline-3-acetic acid; (S)-3-amino-4-(1-naphthyl)-butyric acid; (S)-3-amino-4-(2,4-dichlorophenyl)butyric acid; (S)-3-amino-4-(2-chlorophenyl)-butyric acid; (S)-3-amino-4-(2-cyanophenyl)-butyric acid; (S)-3-amino-4-(2-fluorophenyl)-butyric acid; (S)-3-amino-4-(2-furyl)-butyric acid; (S)-3-amino-4-(2-methylphenyl)-butyric acid; (S)-3-amino-4-(2-naphthyl)-butyric acid; (S)-3-amino-4-(2-thienyl)-butyric acid; (S)-3-amino-4-(2-trifluoromethylphenyl)-butyric acid; (S)-3-amino-4-(3,4-dichlorophenyl)butyric acid; (S)-3-amino-4-(3,4-difluorophenyl)butyric acid; (S)-3-amino-4-(3-benzothienyl)-butyric acid; (S)-3-amino-4-(3-chlorophenyl)-butyric acid; (S)-3-amino-4-(3-cyanophenyl)-butyric acid; (S)-3-amino-4-(3-fluorophenyl)-butyric acid; (S)-3-amino-4-(3-methylphenyl)-butyric acid; (S)-3-amino-4-(3-pyridyl)-butyric acid; (S)-3-amino-4-(3-thienyl)-butyric acid; (S)-3-amino-4-(3-trifluoromethylphenyl)-butyric acid; (S)-3-amino-4-(4-bromophenyl)-butyric acid; (S)-3-amino-4-(4-chlorophenyl) butyric acid; (S)-3-amino-4-(4-cyanophenyl)-butyric acid; (S)-3-amino-4-(4-fluorophenyl) butyric acid; (S)-3-amino-4-(4-iodophenyl)-butyric acid; (S)-3-amino-4-(4-methylphenyl)-butyric acid; (S)-3-amino-4-(4-nitrophenyl)-butyric acid; (S)-3-amino-4-(4-pyridyl)-butyric acid; (S)-3-amino-4-(4-trifluoromethylphenyl)-butyric acid; (S)-3-amino-4-pentafluoro-phenylbutyric acid; (S)-3-amino-5-hexenoic acid; (S)-3-amino-5-hexynoic acid; (S)-3-amino-5-phenylpentanoic acid; (S)-3-amino-6-phenyl-5-hexenoic acid; 1,2,5,6-tetrahydropyridine-3-carboxylic acid; 1,2,5,6-tetrahydropyridine-4-carboxylic acid; 3-amino-3-(2-chlorophenyl)-propionic acid; 3-amino-3-(2-thienyl)-propionic acid; 3-amino-3-(3-bromophenyl)-propionic acid; 3-amino-3-(4-chlorophenyl)-propionic acid; 3-amino-3-(4-methoxyphenyl)-propionic acid; 3-amino-4,4,4-trifluoro-butyric acid; 3-aminoadipic acid; D-β-phenylalanine; β-leucine; L-β-homoalanine; L-β-homoaspartic acid γ-benzyl ester; L-β-homoglutamic acid δ-benzyl ester; L-β-homoisoleucine; L-β-homoleucine; L-β-homomethionine; L-β-homophenylalanine; L-β-homoproline; L-β-homotryptophan; L-β-homovaline; L-Nω-benzyloxycarbonyl-β-homolysine; Nω-L-β-homoarginine; O-benzyl-L-β-homohydroxyproline; O-benzyl-L-β-homoserine; O-benzyl-L-β-homothreonine; O-benzyl-L-β-homotyrosine; γ-trityl-L-β-homoasparagine; I-β-phenylalanine; L-β-homoaspartic acid γ-t-butyl ester; L-β-homoglutamic acid δ-t-butyl ester; L-Nω-β-homolysine; Nδ-trityl-L-β-homoglutamine; Nω-2,2,4,6,7-pentamethyl-dihydrobenzofuran-5-sulfonyl-L-β-homoarginine; O-t-butyl-L-β-homohydroxy-proline; O-t-butyl-L-β-homoserine; O-t-butyl-L-β-homothreonine; O-t-butyl-L-β-homotyrosine; 2-aminocyclopentane carboxylic acid; and 2-aminocyclohexane carboxylic acid.

Examples of amino acid analogs of alanine, valine, glycine, and leucine include α-methoxyglycine; α-allyl-L-alanine; α-aminoisobutyric acid; α-methyl-leucine; β-(1-naphthyl)-D-alanine; β-(1-naphthyl)-L-alanine; β-(2-naphthyl)-D-alanine; β-(2-naphthyl)-L-alanine; β-(2-pyridyl)-D-alanine; β-(2-pyridyl)-L-alanine; β-(2-thienyl)-D-alanine; β-(2-thienyl)-L-alanine; β-(3-benzothienyl)-D-alanine; β-(3-benzothienyl)-L-alanine; β-(3-pyridyl)-D-alanine; β-(3-pyridyl)-L-alanine; β-(4-pyridyl)-D-alanine; β-(4-pyridyl)-L-alanine; β-chloro-L-alanine; β-cyano-L-alanin; β-cyclohexyl-D-alanine; β-cyclohexyl-L-alanine; β-cyclopenten-1-yl-alanine; β-cyclopentyl-alanine; β-cyclopropyl-L-Ala-OH.dicyclohexylammonium salt; β-t-butyl-D-alanine; β-t-butyl-L-alanine; γ-aminobutyric acid; L-α,β-diaminopropionic acid; 2,4-dinitro-phenylglycine; 2,5-dihydro-D-phenylglycine; 2-amino-4,4,4-trifluorobutyric acid; 2-fluoro-phenylglycine; 3-amino-4,4,4-trifluoro-butyric acid; 3-fluoro-valine; 4,4,4-trifluoro-valine; 4,5-dehydro-L-leu-OH.dicyclohexylammonium salt; 4-fluoro-D-phenylglycine; 4-fluoro-L-phenylglycine; 4-hydroxy-D-phenylglycine; 5,5,5-trifluoro-leucine; δ-aminohexanoic acid; cyclopentyl-D-Gly-OH.dicyclohexylammonium salt; cyclopentyl-Gly-OH.dicyclohexylammonium salt; D-α,β-diaminopropionic acid; D-α-aminobutyric acid; D-α-t-butylglycine; D-(2-thienyl)glycine; D-β-thienyl)glycine; D-2-aminocaproic acid; D-2-indanylglycine; D-allylglycine-dicyclohexylammonium salt; D-cyclohexylglycine; D-norvaline; D-phenylglycine; β-aminobutyric acid; β-aminoisobutyric acid; (2-bromophenyl)glycine; (2-methoxyphenyl)glycine; (2-methylphenyl)glycine; (2-thiazoyl)glycine; (2-thienyl)glycine; 2-amino-3-(dimethylamino)-propionic acid; L-α,β-diaminopropionic acid; L-α-aminobutyric acid; L-α-t-butylglycine; L-β-thienyl)glycine; L-2-amino-3-(dimethylamino)-propionic acid; L-2-aminocaproic acid dicyclohexyl-ammonium salt; L-2-indanylglycine; L-allylglycine.dicyclohexyl ammonium salt; L-cyclohexylglycine; L-phenylglycine; L-propargylglycine; L-norvaline; N-α-aminomethyl-L-alanine; D-α,γ-diaminobutyric acid; L-α,γ-diaminobutyric acid; β-cyclopropyl-L-alanine; (N-β-(2,4-dinitrophenyl))-L-α,β-diaminopropionic acid; (N-β-1-(4,4-dimethyl-2,6-dioxocyclohex-1-ylidene)ethyl)-D-α,β-diaminopropionic acid; (N-β-1-(4,4-dimethyl-2,6-dioxocyclohex-1-ylidene)ethyl)-L-α,β-diaminopropionic acid; (N-β-4-methyltrityl)-L-α,β-diaminopropionic acid; (N-β-allyloxycarbonyl)-L-α,β-diaminopropionic acid; (N-γ-1-(4,4-dimethyl-2,6-dioxocyclohex-1-ylidene)ethyl)-D-α,γ-diaminobutyric acid; (N-γ-1-(4,4-dimethyl-2,6-dioxocyclohex-1-ylidene)ethyl)-L-α,γ-diaminobutyric acid; (N-γ-4-methyltrityl)-D-α,γ-diaminobutyric acid; (N-γ-4-methyltrityl)-L-α,γ-diaminobutyric acid; (N-γ-allyloxycarbonyl)-L-α,γ-diaminobutyric acid; D-α,γ-diaminobutyric acid; 4,5-dehydro-L-leucine; cyclopentyl-D-Gly-OH; cyclopentyl-Gly-OH; D-allylglycine; D-homocyclohexylalanine; L-1-pyrenylalanine; L-2-aminocaproic acid; L-allylglycine; L-homocyclohexylalanine; and N-(2-hydroxy-4-methoxy-Bzl)-Gly-OH.

Examples of amino acid analogs of arginine and lysine include citrulline; L-2-amino-3-guanidinopropionic acid; L-2-amino-3-ureidopropionic acid; L-citrulline; Lys(Me)2-OH; Lys(N3)-0H; Nδ-benzyloxycarbonyl-L-ornithine; Nω-nitro-D-arginine; Nω-nitro-L-arginine; α-methyl-ornithine; 2,6-diaminoheptanedioic acid; L-ornithine; (Nδ-1-(4,4-dimethyl-2,6-dioxo-cyclohex-1-ylidene)ethyl)-D-ornithine; (Nδ-1-(4,4-dimethyl-2,6-dioxo-cyclohex-1-ylidene)ethyl)-L-ornithine; (Nδ-4-methyltrityl)-D-ornithine; (Nδ-4-methyltrityl)-L-ornithine; D-ornithine; L-ornithine; Arg(Me)(Pbf)-OH; Arg(Me)2-OH (asymmetrical); Arg(Me)2-OH (symmetrical); Lys(ivDde)-OH; Lys(Me)2-OH.HCl; Lys(Me3)-OH chloride; Nω-nitro-D-arginine; and Nω-nitro-L-arginine.

Examples of amino acid analogs of aspartic and glutamic acids include α-methyl-D-aspartic acid; α-methyl-glutamic acid; α-methyl-L-aspartic acid; γ-methylene-glutamic acid; (N-γ-ethyl)-L-glutamine; [N-α-(4-aminobenzoyl)]-L-glutamic acid; 2,6-diaminopimelic acid; L-α-aminosuberic acid; D-2-aminoadipic acid; D-α-aminosuberic acid; α-aminopimelic acid; iminodiacetic acid; L-2-aminoadipic acid; threo-β-methyl-aspartic acid; γ-carboxy-D-glutamic acid γ,γ-di-t-butyl ester; γ-carboxy-L-glutamic acid γ,γ-di-t-butyl ester; Glu(Oall)-OH; L-Asu(OtBu)-OH; and pyroglutamic acid.

Examples of amino acid analogs of cysteine and methionine include

Cys(farnesyl)-OH, Cys(farnesyl)-Ome, α-methyl-methionine, Cys(2-hydroxyethyl)-OH, Cys(3-aminopropyl)-0H, 2-amino-4-(ethylthio)butyric acid, buthionine, buthioninesulfoximine, ethionine, methionine methylsulfonium chloride, selenomethionine, cysteic acid, [2-(4-pyridyl)ethyl]-DL-penicillamine, [2-(4-pyridyl)ethyl]-L-cysteine, 4-methoxybenzyl-D-penicillamine, 4-methoxybenzyl-L-penicillamine, 4-methylbenzyl-D-penicillamine, 4-methylbenzyl-L-penicillamine, benzyl-D-cysteine, benzyl-L-cysteine, benzyl-DL-homocysteine, carbamoyl-L-cysteine, carboxyethyl-L-cysteine, carboxymethyl-L-cysteine, diphenylmethyl-L-cysteine, ethyl-L-cysteine, methyl-L-cysteine, t-butyl-D-cysteine, trityl-L-homocysteine, trityl-D-penicillamine, cystathionine, homocystine, L-homocystine, (2-aminoethyl)-L-cysteine, seleno-L-cystine, cystathionine, Cys(StBu)-OH, and acetamidomethyl-D-penicillamine.

Examples of amino acid analogs of phenylalanine and tyrosine include β-methyl-phenylalanine, β-hydroxyphenylalanine, α-methyl-3-methoxy-DL-phenylalanine, α-methyl-D-phenylalanine, α-methyl-L-phenylalanine, 1,2,3,4-tetrahydroisoquinoline-3-carboxylic acid, 2,4-dichloro-phenylalanine, 2-(trifluoromethyl)-D-phenylalanine, 2-(trifluoromethyl)-L-phenylalanine, 2-bromo-D-phenylalanine, 2-bromo-L-phenylalanine, 2-chloro-D-phenylalanine, 2-chloro-L-phenylalanine, 2-cyano-D-phenylalanine, 2-cyano-L-phenylalanine, 2-fluoro-D-phenylalanine, 2-fluoro-L-phenylalanine, 2-methyl-D-phenylalanine, 2-methyl-L-phenylalanine, 2-nitro-D-phenylalanine, 2-nitro-L-phenylalanine, 2;4;5-trihydroxy-phenylalanine, 3,4,5-trifluoro-D-phenylalanine, 3,4,5-trifluoro-L-phenylalanine, 3,4-dichloro-D-phenylalanine, 3,4-dichloro-L-phenylalanine, 3,4-difluoro-D-phenylalanine, 3,4-difluoro-L-phenylalanine, 3,4-dihydroxy-L-phenylalanine, 3,4-dimethoxy-L-phenylalanine, 3,5,3′-triiodo-L-thyronine, 3,5-diiodo-D-tyrosine, 3,5-diiodo-L-tyrosine, 3,5-diiodo-L-thyronine, 3-(trifluoromethyl)-D-phenylalanine, 3-(trifluoromethyl)-L-phenylalanine, 3-amino-L-tyrosine, 3-bromo-D-phenylalanine, 3-bromo-L-phenylalanine, 3-chloro-D-phenylalanine, 3-chloro-L-phenylalanine, 3-chloro-L-tyrosine, 3-cyano-D-phenylalanine, 3-cyano-L-phenylalanine, 3-fluoro-D-phenylalanine, 3-fluoro-L-phenylalanine, 3-fluoro-tyrosine, 3-iodo-D-phenylalanine, 3-iodo-L-phenylalanine, 3-iodo-L-tyrosine, 3-methoxy-L-tyrosine, 3-methyl-D-phenylalanine, 3-methyl-L-phenylalanine, 3-nitro-D-phenylalanine, 3-nitro-L-phenylalanine, 3-nitro-L-tyrosine, 4-(trifluoromethyl)-D-phenylalanine, 4-(trifluoromethyl)-L-phenylalanine, 4-amino-D-phenylalanine, 4-amino-L-phenylalanine, 4-benzoyl-D-phenylalanine, 4-benzoyl-L-phenylalanine, 4-bis(2-chloroethyl)amino-L-phenylalanine, 4-bromo-D-phenylalanine, 4-bromo-L-phenylalanine, 4-chloro-D-phenylalanine, 4-chloro-L-phenylalanine, 4-cyano-D-phenylalanine, 4-cyano-L-phenylalanine, 4-fluoro-D-phenylalanine, 4-fluoro-L-phenylalanine, 4-iodo-D-phenylalanine, 4-iodo-L-phenylalanine, homophenylalanine, thyroxine, 3,3-diphenylalanine, thyronine, ethyl-tyrosine, and methyl-tyrosine.

Examples of amino acid analogs of proline include 3,4-dehydro-proline, 4-fluoro-proline, cis-4-hydroxy-proline, thiazolidine-2-carboxylic acid, and trans-4-fluoro-proline.

Examples of amino acid analogs of serine and threonine include 3-amino-2-hydroxy-5-methylhexanoic acid, 2-amino-3-hydroxy-4-methylpentanoic acid, 2-amino-3-ethoxybutanoic acid, 2-amino-3-methoxybutanoic acid, 4-amino-3-hydroxy-6-methylheptanoic acid, 2-amino-3-benzyloxypropionic acid, 2-amino-3-benzyloxypropionic acid, 2-amino-3-ethoxypropionic acid, 4-amino-3-hydroxybutanoic acid, and α-methylserine.

Examples of amino acid analogs of tryptophan include α-methyl-tryptophan; β-(3-benzothienyl)-D-alanine; β-(3-benzothienyl)-L-alanine; 1-methyl-tryptophan; 4-methyl-tryptophan; 5-benzyloxy-tryptophan; 5-bromo-tryptophan; 5-chloro-tryptophan; 5-fluoro-tryptophan; 5-hydroxy-tryptophan; 5-hydroxy-L-tryptophan; 5-methoxy-tryptophan; 5-methoxy-L-tryptophan; 5-methyl-tryptophan; 6-bromo-tryptophan; 6-chloro-D-tryptophan; 6-chloro-tryptophan; 6-fluoro-tryptophan; 6-methyl-tryptophan; 7-benzyloxy-tryptophan; 7-bromo-tryptophan; 7-methyl-tryptophan; D-1,2,3,4-tetrahydro-norharman-3-carboxylic acid; 6-methoxy-1,2,3,4-tetrahydronorharman-1-carboxylic acid; 7-azatryptophan; L-1,2,3,4-tetrahydro-norharman-3-carboxylic acid; 5-methoxy-2-methyl-tryptophan; and 6-chloro-L-tryptophan.

In some embodiments, modified netrin-1 peptides may comprise one or more non-essential amino acids. A non-essential amino acid residue can be a residue that can be altered from the wild-type sequence of a polypeptide without abolishing or substantially altering its essential biological or biochemical activity (e.g., receptor binding or activation).

In some embodiments, modified netrin-1 peptides may comprise one or more conservative amino acid substitutions. In some embodiments, a conservative amino acid substitution is one in which the amino acid residue is replaced with an amino acid residue having a side chain. Amino acids with basic side chains include Arg, His, and Lys amino acids with acidic side chains include Asp and Glu, amino acids with uncharged polar side chains include Asn, Cys, Gln, Gly, Ser, Thr, and Tyr, amino acids with nonpolar side chains include Ala, Ile, Leu, Met, Phe, Pro, Trp, and Val, amino acids with I-branched side chains include Ile, Thr, and Val, and amino acids with aromatic side chains include His, Phe, Trp, and Tyr. In some embodiments, a conservative amino acid substitution is a very highly conserved substitution, a highly conserved substitution, or a conserved substitution as set forth in the following table:

| | Very Highly - | | |
|---|---|---|---|
| Original | Conserved | Highly Conserved Substitutions | Conserved Substitutions |
| Residue | Substitutions | (from the Blosum90 Matrix) | (from the Blosum65 Matrix) |
| Ala | Ser | Gly, Ser, Thr | Cys, Gly, Ser, Thr, Val |
| Arg | Lys | Gln, His, Lys | Asn, Gln, Glu, His, Lys |
| Asn | Gln; His | Asp, Gln, His, Lys, Ser, Thr | Arg, Asp, Gln, Glu, His, Lys, Ser, Thr |
| Asp | Glu | Asn, Glu | Asn, Gln, Glu, Ser |
| Cys | Ser | None | Ala |
| Gln | Asn | Arg, Asn, Glu, His, Lys, Met | Arg, Asn, Asp, Glu, His, Lys, Met, Ser |
| Glu | Asp | Asp, Gln, Lys | Arg, Asn, Asp, Gln, His, Lys, Ser |
| Gly | Pro | Ala | Ala, Ser |
| His | Asn; Gln | Arg, Asn, Gln, Tyr | Arg, Asn, Gln, Glu, Tyr |
| Ile | Leu; Val | Leu, Met, Val | Leu, Met, Phe, Val |
| Leu | Ile; Val | Ile, Met, Phe, Val | Ile, Met, Phe, Val |
| Lys | Arg; Gln; Glu | Arg, Asn, Gln, Glu | Arg, Asn, Gln, Glu, Ser, |
| Met | Leu; Ile | Gln, Ile, Leu, Val | Gln, Ile, Leu, Phe, Val |
| Phe | Met; Leu; Tyr | Leu, Trp, Tyr | Ile, Leu, Met, Trp, Tyr |
| Ser | Thr | Ala, Asn, Thr | Ala, Asn, Asp, Gln, Glu, Gly, Lys, Thr |
| Thr | Ser | Ala, Asn, Ser | Ala, Asn, Ser, Val |
| Trp | Tyr | Phe, Tyr | Phe, Tyr |
| Tyr | Trp; Phe | His, Phe, Trp | His, Phe, Trp |
| Val | Ile; Leu | Ile, Leu, Met | Ala, Ile, Leu, Met, Thr |

The modified netrin-1 peptides are advantageous over the full-length netrin-1 protein because they are shorter in size, and as a result, may be 1) conveniently and successfully produced in large quantities, 2) more affordable, 3) activate the protective pathway with less or reduced side effects as compared to the full-length netrin-1, 4) easier to deliver to the target organ in a subject, and 5) more stable and active during the treatment to have bigger protective effects. The modified netrin-1 peptides are advantageous over prior art peptides because the modified netrin-1 peptides exhibit superior activity over the prior art peptides.

Based on the sequence similarity and differences between the modified netrin-1 peptides exemplified herein and the prior art peptides, it is believed that the substitution of the first amino acid residue (X1 of SEQ ID NO: 1 as described herein, and the first cysteine, i.e., amino acid residue at position 1 of the “core sequence” of the prior art peptides) is responsible for conferring the superior activity of the modified netrin-1 peptides. The superior activity conferred by the X1 amino acid position is unexpected because those skilled in the art recognize serine as being a very highly conserved substitution for cysteine. As such, one skilled in the art would expect that modifying the prior art peptide, V1, to have a serine instead of a cysteine at amino acid position 1, would result in substantially similar activity. However, as set forth in Table 3, replacing the cysteine with serine results in almost a 30% ((71-55)/55=29%) increase in the reduction of infarct size.

Because modified netrin-1 peptides are shown to significantly reduce infarct size of cardiac tissue, one or more modified netrin-1 peptides may be used to treat acute myocardial infarction in subjects. In some embodiments, one or more modified netrin-1 peptides may be used to treat, inhibit, or reduce ischemia/reperfusion injury to cardiac tissue in a subject. As used herein, the term “ischemia/reperfusion injury” (FR injury) refers to an injury of an organ, e.g., heart, caused by putting the organ into an ischemic condition such as by thomboembolic events, surgery, or cardiac standstill. Clinically relevant situations include occlusion of coronary arteries/branches that happen during myocardial infarction (ischemia). Treatment with percutaneous transluminal coronary angioplasty (PTCA) procedure creates a reperfusion condition that is known to cause additional injury that can be however protected by pharmacological post-conditioning (administering modified netrin-1 peptides at the reperfusion stage). Therefore, in some embodiments, the present invention is also directed towards acute treatment of myocardial infarction by administering one or more modified netrin-1 peptides (e.g., intravenously) alone or in combination with PTCA/drug eluting stent. In some embodiments, the modified netrin-1 peptides may be used to reduce or inhibit the infarct size of cardiac tissue and/or treat, inhibit, or reduce damage to cardiac tissue resulting from myocardial infarction.

Because modified netrin-1 peptides reduce infarct size of cardiac tissue like the prior art peptides, except the amount of the reduction is significantly more than that observed for the prior art peptides, the modified netrin-1 peptides are expected to induce phosphorylation of ERK1/2, eNOSₛ₁₁₇₇, and/or eNOSₛ₁₁₇₉, and induce nitric oxide production to at least the same degree as the prior art peptides. Therefore, in some embodiments, one or more modified netrin-1 peptides are used to induce phosphorylation of ERK1/2, eNOSₛ₁₁₇₇, and/or eNOSₛ₁₁₇₉ in subjects. Similarly, it is believed that the modified netrin-1 peptides can induce nitric oxide production to at least the same degree as the prior art peptides. Therefore, in some embodiments, one or more modified netrin-1 peptides are used to increase nitric oxide production in subjects.

### Peptide Compositions and Delivery

Administration of one or more modified netrin-1 peptides can be accomplished by direct administration or accomplished by administering one or more nucleic acid molecules which encode the one or more modified netrin-1 peptides.

In some embodiments, a therapeutically effective amount of one or more of the peptides fragments according to the present invention are administered to a subject. As used herein, a “therapeutically effective amount” refers to an amount that may be used to treat, alleviate, ameliorate, prevent, or inhibit a given disease or condition, such as an I/R injury, or a symptom thereof, in a subject as compared to a control, such as a placebo. For example, in some embodiments, a therapeutically effective amount is an amount which has a beneficial effect in a subject having signs and/or symptoms of I/R injury of cardiac tissue. In some embodiments, a therapeutically effective amount is an amount which inhibits or reduces signs and/or symptoms of I/R injury as compared to a control. Signs and symptoms of FR injury to cardiac tissue are known in the art and include sudden chest pain (typically radiating to the left arm or left side of the neck), shortness of breath, nausea, vomiting, palpitations, sweating, and anxiety. In some embodiments, the therapeutically effective amount is one which is sufficient to increase phosphorylation of ERK1/2, eNOS, or both and/or to increase nitric oxide production, in a subject as compared to a control. In some embodiments, a therapeutically effective amount is one which is sufficient to activate cardioprotective mechanisms in a subject as compared to a control. In some embodiments, the therapeutically effective amount of one or more modified netrin-1 peptides range from about 1 ng/kg to about 100 mg/kg body weight, about 0.001 mg/kg to about 100 mg/kg body weight, about 0.01 mg/kg to about 10 mg/kg body weight, about 0.01 mg/kg to about 5 mg/kg body weight, about 0.01 mg/kg to about 3 mg/kg body weight, about 0.01 mg/kg to about 2 mg/kg, about 0.01 mg/kg to about 1 mg/kg, or about 0.01 mg/kg to about 0.5 mg/kg body weight. In some embodiments, about 5 mg/kg to about 10 mg/kg body weight of one or more modified netrin-1 peptides are administered to the subject at the time of or immediately after ischemia and reperfusion or an I/R injury in a subject. In some embodiments, about 1 ng/kg to about 25 ng/kg, preferably about 10 ng/kg to about 20 ng/kg, and more preferably about 15 ng/kg body weight of one or more modified netrin-1 peptides are administered daily for a given period of time, e.g., about 4 weeks, to treat, prevent, or inhibit post-angioplasty restenosis.

It should be noted that treatment of a subject with a therapeutically effective amount may be administered as a single dose or as a series of several doses. The dosages used for treatment may increase or decrease over the course of a given treatment. Optimal dosages for a given set of conditions and a given subject may be ascertained by those skilled in the art using dosage-determination tests and/or diagnostic assays in the art. Dosage-determination tests and/or diagnostic assays may be used to monitor and adjust dosages during the course of treatment. In some embodiments, the one or more netrin-1 compounds are administered in the form of a composition.

In some embodiments, the compositions comprise, consist essentially of, or consist of one or more modified netrin-1 peptides. As used herein, a composition “comprising” one or more modified netrin-1 peptides means that the composition may contain other compounds, including proteins that are not modified netrin-1 peptides (e.g., prior art peptides). As used herein, a composition “consisting essentially of” one or more modified netrin-1 peptides means that the composition may comprise proteins in addition to the modified netrin-1 peptides so long as the additional proteins do not materially change the activity or function of the modified netrin-1 peptides that are contained in the composition. As used herein, a composition “consisting of” one or more modified netrin-1 peptides means that the composition does not contain proteins in addition to the one or more modified netrin-1 peptides. Compositions consisting of one or more modified netrin-1 peptides may comprise ingredients other than proteins, e.g., pharmaceutically acceptable carriers, surfactants, preservatives, etc. In some embodiments, compositions consisting of one or more modified netrin-1 peptides may contain insignificant amounts of contaminants, which may include peptide contaminants, e.g., smaller fragments of the one or more modified netrin-1 peptides, which may result from, for example, the synthesis of the one or more modified netrin-1 peptides, subsequent processing, storage conditions, and/or protein degradation.

In some embodiments, the compositions may comprise, consist essentially of, or consist of one or more purified modified netrin-1 peptides. As used herein, a “purified” modified netrin-1 peptide means that an amount of the macromolecular components that are naturally associated with the modified netrin-1 peptide have been removed from the modified netrin-1 peptide. As used herein, a composition comprising, consisting essentially of, or consisting of one or more purified modified netrin-1 peptides means that the composition does not contain an amount of the macromolecular components that are naturally associated with the one or more modified netrin-1 peptides and/or the reagents used to synthesize the modified netrin-1 peptides. In some embodiments, the amount removed from the one or more modified netrin-1 peptides (or is not present in the composition) is at least about 60%, at least about 70%, at least about 75%, at least about 80%, at least about 85%, at least about 90%, at least about 95%, at least about 96%, at least about 96%, at least about 97%, at least about 98%, at least about 99%, or 100% of the macromolecular components and/or reagents. In some embodiments, the composition is free of at least about 60%, at least about 70%, at least about 75%, at least about 80%, at least about 85%, at least about 90%, at least about 95%, at least about 96%, at least about 96%, at least about 97%, at least about 98%, at least about 99%, or 100% of the macromolecular components naturally associated with the one or more modified netrin-1 peptides and/or the reagents used to synthesize the one or more modified netrin-1 peptides. In some embodiments, the compositions of the present invention consist solely of one or more modified netrin-1 peptides, e.g., the one or more modified netrin-1 peptides in a solid or crystalized form.

In some embodiments, compositions according to the present invention include one or more modified netrin-1 peptides and a pharmaceutically acceptable carrier. The term “pharmaceutically acceptable carrier” as used herein refers to a carrier or diluent, which are added to a composition by the hand of man, that is generally non-toxic to an intended recipient and does not significantly inhibit activity of the one or more modified netrin-1 peptides included in the composition. In some embodiments, compositions according to the present invention may include one or more excipients, diluents, auxiliaries, preservatives, solubilizing agents, buffers, thickening agents, gelling agents, foaming agents, surfactants, binders, suspending agents, disintegrating agents, wetting agents, solvents, plasticizers, fillers, colorants, dispersants, flavoring agents, and/or the like known in the art.

A composition according to the present invention generally includes about 0.1-99% of one or more modified netrin-1 peptides. In some embodiments, a composition according to the present invention includes one or more modified netrin-1 peptides and one or more prior art peptides. In some embodiments, a composition according to the present invention includes one or more modified netrin-1 peptides and full-length netrin-1. In some embodiments, the compositions are synergistic compositions, e.g., compositions comprising a modified netrin-1 peptide according to the present invention and a second protein which may be a second modified netrin-1 peptide according to the present invention or, for example, a full-length netrin-1 protein or a prior art peptide, in synergistic amounts.

In some embodiments, one or more modified netrin-1 peptides are included in a composition of the present invention in the form of a free acid or free base. In some embodiments, one or more modified netrin-1 peptides are included in a composition in the form of a pharmaceutically acceptable salt such as an acid or base addition salt. A pharmaceutically acceptable salt refers to any salt form of the one or more modified netrin-1 peptides that is generally non-toxic to an intended recipient and does not significantly inhibit activity of the one or more modified netrin-1 peptides or other active agent included in the composition. In some embodiments, the one or more modified netrin-1 peptides are provided in the form of a hydrate or a prodrug.

A composition including one or more modified netrin-1 peptides may be administered by a systemic route and/or by a local route. Suitable routes of administration illustratively include intravenous, oral, buccal, parenteral, intrathecal, intracerebroventricular, intraperitoneal, intracardiac, intraarterial, intravesicle, ocular, intraocular, rectal, vaginal, subcutaneous, intradermal, transdermal, intramuscular, topical, intranasal, and transmucosal. In some embodiments, the one or more modified netrin-1 peptides and compositions thereof are administered intravenously or by intraventricular injection, e.g., during angioplasty for acute MI treatment or open heart surgery.

In some embodiments, the modified netrin-1 peptides and compositions according to the present invention may be modified using methods and compositions known in the art to improve their biological half-life, stability, efficacy, bioavailability, bioactivity, or a combination thereof.

For example, in some embodiments, the modified netrin-1 peptides may be subjected to cyclization to result in a cyclic peptide which is resistant to proteolytic degradation. Cyclization may be carried out between side chains or ends of the peptide sequences through disulfide bonds, lanthionine, dicarba, hydrazine, or lactam bridges using methods known in the art.

In some embodiments, the modified netrin-1 peptides may be conjugated to a molecule such as vitamin B12, a lipid, or an ethylene oxide compound, e.g., polyethylene glycol (PEG), polyethylene oxide (PEO), and polyoxyethylene (POE), methoxypolyethylene glycol (MPEG), monomethoxypolyethylene glycol (mPEG), diethylene glycol (mini-PEG), and the like. The ethylene oxide compound may be further functionalized with, for example, amine binding terminal functional groups such as N-hydroxysuccinimide esters, N-hydroxysuccinimide carbonates, and aliphatic aldehyde, or thiol binding groups such as maleimide, pyridyl disulphides, and vinyl sulfonates. Since amino groups (α-amino and ε-lysine amino) and cysteine residues are well suited for conjugation, the modified netrin-1 peptides may further include one or more amino acid residues for conjugation to an ethylene oxide molecule or a carrier compound known in the art. The pharmacokinetic and pharmacodynamic properties of a conjugated peptide may be further modified by the use of a particular linker. For example, propyl and amyl linkers can be used to provide a conjugate having a loose conformation whereas a phenyl linker may be used to provide a denser conformation as well as shield domains adjacent to the C-terminus. It is noted that dense conformations are generally more efficient in maintaining bioactivity, prolonging plasma half-life, lowering proteolytic sensitivity, and immunogenicity relative to loose conformations.

In some embodiments, the modified netrin-1 peptides may be hyperglycosylated using methods known in the art, e.g., in situ chemical reactions or site-directed mutagenesis. Hyperglycosylation may result in either N-linked or O-linked protein glycosylation. The clearance rate of a given modified netrin-1 peptide may be optimized by the selection of the particular saccharide. For example, polysialic acid (PSA) is available in different sizes and its clearance depends on type and molecular size of the polymer. Thus, for example, PSAs having high molecular weights may be suitable for the delivery of low-molecular-weight modified netrin-1 peptides, and PSAs having low molecular weights may be suitable for the delivery of modified netrin-1 peptides having high molecular weights. The type of saccharide can be used to target the modified netrin-1 peptide to a particular tissue or cell. For example, modified netrin-1 peptides conjugated with mannose can be recognized by mannose-specific lectins, e.g., mannose receptors and mannan-binding proteins, and are taken up by the liver. In some embodiments, the modified netrin-1 peptides may be hyperglycosylated to improve their physical and chemical stability under different environmental conditions, e.g., to inhibit inactivation under stress conditions and reduce aggregation resulting from production and storage conditions.

In some embodiments, a drug delivery system, such as microparticles, nanoparticles (particles having sizes ranging from 10 to 1000 nm), nanoemulsions, liposomes, and the like, may be used to provide protection of sensitive proteins, prolong release, reduce administration frequency, increase patient compliance, and control plasma levels. Various natural or synthetic microparticles and nanoparticles, which may be biodegradable and/or biocompatible polymers, may be used. Microparticles and nanoparticles can be fabricated from lipids, polymers, and/or metal. Polymeric microparticles and nanoparticles may be fabricated from natural or synthetic polymers, such as starch, alginate, collagen, chitosan, polycaprolactones (PCL), polylactic acid (PLA), poly (lactide-co-glycolide) (PLGA), and the like. In some embodiments, the nanoparticles are solid lipid nanoparticles (SLNs), carbon nanotubes, nanospheres, nanocapules, and the like. In some embodiments, the polymers are hydrophilic. In some embodiments, the polymers are thiolated polymers.

Since the rate and extent of drug release from microparticles and nanoparticles may depend on the composition of polymer and fabrication methods one may select a given composition and fabrication method, e.g., spray drying, lyophilization, microextrusion, and double emulsion, to confer a desired drug release profile. Since peptide fragments incorporated in or on microparticles or nanoparticles may be prone to denaturation at aqueous-organic interface during formulation development, different stabilizing excipients and compositions can be used to prevent aggregation and denaturation. For example, PEG and sugars, e.g., PEG (MW 5000) and maltose with α-chymotrypsin, may be added to the composition to reduce aggregation and denaturation. Additionally, chemically modified peptide fragments, e.g., conjugated peptide fragments and hyperglycosylated peptide fragments, may be employed.

Protein stability can also be achieved by the selected fabrication method. For example, to prevent degradation at aqueous-organic interface, non-aqueous methodology called ProLease® technology may be used. Peptide fragments in solid state can also be encapsulated using solid-in-oil-in-water (s/o/w) methods, e.g., spray- or spray-freeze-dried peptide fragments or peptide-loaded solid nanoparticles can be encapsulated in microspheres using s/o/w methods. Hydrophobic ion-pairing (HIP) complexation may be used to enhance protein stability and increase encapsulation efficiency into microparticles and nanoparticles. In hydrophobic ion-pairing (HIP) complexation, ionizable functional groups of a peptide are complexed with ion-pairing agents (e.g., surfactant or polymer) containing oppositely charged functional groups leading to formation of HIP complex where hydrophilic protein molecules exist in a hydrophobic complex form.

In some embodiments, liposomes of either synthetic or natural origin and various sizes, e.g., 20 nm to several hundred micrometers, may be used to deliver peptide fragments. Depending on the preparation method, the liposomes can be small unilamellar vesicles (25-50 nm), large unilamellar vesicles (100-200 nm), giant unilamellar vesicles (1-2 μm), and multilamellar vesicles (MLV; 1 μm-2 μm). The peptide fragments being delivered can be either encapsulated into liposomes or adsorbed on the surface. The size and surface properties of liposomes may be optimized for a desired result. For example, unilamellar and multilamellar liposomes provide sustained release from several hours to days after intravascular administration. The prolonged drug release can be achieved by multivesicular liposomes, also known as DepoFoam® technology. Unlike ULV and MLV, multivesicular liposomes are composed of nonconcentric multiple aqueous chambers surrounded by a network of lipid layers which confers an increased level of stability and longer duration of drug release. The liposomes may be further modified to achieve a desired result. For example, the liposomes may be PEGylated or have other surface modifications in order to interfere with recognition and uptake by the reticuloendothelial system and provide increased circulation times.

Exemplary liposomes suitable for use according to the present invention include multilamellar vesicles (MLV), oligolamellar vesicles (OLV), unilamellar vesicles (UV), small unilamellar vesicles (SUV), medium-sized unilamellar vesicles (MUV), large unilamellar vesicles (LUV), giant unilamellar vesicles (GUV), multivesicular vesicles (MVV), single or oligolamellar vesicles made by reverse-phase evaporation method (REV), multilamellar vesicles made by the reverse-phase evaporation method (MLV-REV), stable plurilamellar vesicles (SPLV), frozen and thawed MLV (FATMLV), vesicles prepared by extrusion methods (VET), vesicles prepared by French press (FPV), vesicles prepared by fusion (FUV), dehydration-rehydration vesicles (DRV), and bubblesomes (BSV).

The liposomes may comprise additional lipids, e.g., carrier lipids, including palmitoylphosphatidylcholine (DPPC), phosphatidylcholine (PC; lecithin), phosphatidic acid (PA), phosphatidylglycerol (PG), phosphatidylethanolamine (PE), phosphatidylserine (PS), di stearoylphosphatidylcholine (DSPC), dimyristoylphosphatidylcholine (DMPC), dipalmitoylphosphatidyglycerol (DPPG), di stearoylphosphatidyglycerol (DSPG), dimyristoylphosphatidylglycerol (DMPG), dipalmitoylphosphatidic acid (DPPA); dimyristoylphosphatidic acid (DMPA), di stearoylphosphatidic acid (DSPA), dipalmitoylphosphatidylserine (DPPS), dimyristoylphosphatidylserine (DMPS), distearoylphosphatidylserine (DSPS), dipalmitoylphosphatidyethanolamine (DPPE), dimyristoylphosphatidylethanolamine (DMPE), distearoylphosphatidylethanolamine (DSPE), and the like, or combinations thereof. In some embodiments, the liposomes further comprise a sterol (e.g., cholesterol).

In some embodiments, micelles may be used to deliver the modified netrin-1 peptides. Phospholipids such as DSPE-PEG, co-polymeric systems PEG-PE, PLA-PEG and hyperbranched poly([amine-ester]-co-[d,l-lactide]) and polyion complexes may be used to increase stability and pharmacokinetics.

Thermosensitive gels may be used to deliver the modified netrin-1 peptides. Thermoreversible block copolymers comprising PEG, PCL, PLA, poly(glycolide), PLGA, poly (N-isopropylacrylamide), polyethylene oxide, chitosan, and the like may be used to provide controlled release of the peptide fragments. Examples of thermosensitive gels include PLGA-PEG-PLGA triblock copolymer gels and Pluronic F-127 (PF127). Polyelectrolyte complexes and/or PEGylation may be used to provide sustained release of proteins from the gels. Microparticles and/or nanoparticles may also be used in combination with gels to provide sustained drug delivery.

Modified netrin-1 peptides may be chemically synthesized, or recombinantly expressed in a cell system or a cell-free system. Synthetic methods include liquid-phase synthesis, solid-phase synthesis, and microwave assisted peptide synthesis. The peptide fragments may be modified by acylation, alkylation, amidation, arginylation, polyglutamylation, polyglycylation, butyrylation, gamma-carboxylation, glycosylation, malonylation, hydroxylation, iodination, nucleotide addition (e.g., ADP-ribosylation), oxidation, phosphorylation, adenylylation, propionylation, S-glutathionylation, S-nitrosylation, succinylation, sulfation, glycation, palmitoylation, myristoylation, isoprenylation or prenylation (e.g., farnesylation or geranylgeranylation), glypiation, lipoylation, attachement of flavin moiety (e.g., FMN or FAD), attachment of heme C, phosphopantetheinylation, retinylidene Schiff base formation, diphthamide formation, ethanolamine phosphoglycerol attachment, hypusine formuation, biotinylation, pegylation, ISGylation, SUMUylation, ubiquitination, Neddylation, Pupylation, citrullination, deamidation, eliminylation, carbamylation, or a combination thereof.

Compositions comprising one or more modified netrin-1 peptides may be subjected to one or more rounds of purification or concentration steps known in the art to remove impurities and/or concentrate the peptide fragments. Thus, in some embodiments, the present invention provides peptide compositions having a purity and/or composition not found in nature. In some cases, the peptide composition is at most 30%, 40%, 50%, 60%, 70%, 80%, 90%, 95%, 99%, 99.9%, or 100% pure peptide fragments. In some cases, the peptide composition is at least 30%, 40%, 50%, 60%, 70%, 80%, 90%, 95%, 99%, 99.9%, or 100% pure peptide fragments. In some cases, the composition is free of impurities. In some cases, the amount of the peptide fragments in the peptide composition is at most 30%, 40%, 50%, 60%, 70%, 80%, 90%, 95%, 99%, 99.9%, or 100% weight of the total composition. In some cases, the amount of the peptide fragments in the peptide composition is at least 30%, 40%, 50%, 60%, 70%, 80%, 90%, 95%, 99%, 99.9%, or 100% by weight of the total composition.

Compositions of the present invention include pharmaceutical compositions that comprise one or more modified netrin-1 peptides. The term “pharmaceutical composition” refers to a composition suitable for pharmaceutical use in a subject. A pharmaceutical composition generally comprises an effective amount of an active agent, e.g., one or more modified netrin-1 peptides according to the present invention, and a pharmaceutically acceptable carrier. The term “effective amount” refers to a dosage or amount sufficient to produce a desired result. The desired result may comprise an objective or subjective improvement in the recipient of the dosage or amount, e.g., long-term survival, effective prevention of a disease state, and the like. In some embodiments, the “effective amount” is less than a therapeutically effective amount. In some embodiments, pharmaceutical compositions comprise one or more netrin-1 compounds in a therapeutically effective amount. Pharmaceutical compositions according to the present invention may further include one or more supplementary agents.

One or more modified netrin-1 peptides according to the present invention may be administered, preferably in the form of pharmaceutical compositions, to a subject. Preferably the subject is mammalian, more preferably, the subject is human. Preferred pharmaceutical compositions are those comprising at least one modified netrin-1 peptide in a therapeutically effective amount and a pharmaceutically acceptable vehicle.

Pharmaceutical compositions of the present invention may be formulated for the intended route of delivery, including intravenous, intramuscular, intra peritoneal, subcutaneous, intraocular, intrathecal, intraarticular, intrasynovial, cisternal, intrahepatic, intralesional injection, intracranial injection, infusion, and/or inhaled routes of administration using methods known in the art. Pharmaceutical compositions according to the present invention may include one or more of the following: pH buffered solutions, adjuvants (e.g., preservatives, wetting agents, emulsifying agents, and dispersing agents), liposomal formulations, nanoparticles, dispersions, suspensions, or emulsions, as well as sterile powders for reconstitution into sterile injectable solutions or dispersions. The compositions and formulations of the present invention may be optimized for increased stability and efficacy using methods in the art. See, e.g., Carra et al. (2007) Vaccine 25:4149-4158.

The compositions of the present invention may be administered to a subject by any suitable route including oral, transdermal, subcutaneous, intranasal, inhalation, intramuscular, and intravascular administration. It will be appreciated that the preferred route of administration and pharmaceutical formulation will vary with the condition and age of the subject, the nature of the condition to be treated, the therapeutic effect desired, and the particular modified netrin-1 peptide used.

As used herein, a “pharmaceutically acceptable vehicle” or “pharmaceutically acceptable carrier” are used interchangeably and refer to solvents, dispersion media, coatings, antibacterial and antifungal agents, isotonic and absorption delaying agents, and the like, that are compatible with pharmaceutical administration and comply with the applicable standards and regulations, e.g., the pharmacopeial standards set forth in the United States Pharmacopeia and the National Formulary (USP-NF) book, for pharmaceutical administration. Thus, for example, unsterile water is excluded as a pharmaceutically acceptable carrier for, at least, intravenous administration. Pharmaceutically acceptable vehicles include those known in the art. See, e.g., Remington: The Science and Practice of Pharmacy. 20th ed. (2000) Lippincott Williams & Wilkins. Baltimore, Md.

The pharmaceutical compositions of the present invention may be provided in dosage unit forms. As used herein, a “dosage unit form” refers to physically discrete units suited as unitary dosages for the subject to be treated; each unit containing a predetermined quantity of the one or more modified netrin-1 peptides calculated to produce the desired therapeutic effect in association with the required pharmaceutically acceptable carrier. The specification for the dosage unit forms of the invention are dictated by and directly dependent on the unique characteristics of the given modified netrin-1 peptide and desired therapeutic effect to be achieved, and the limitations inherent in the art of compounding such an active compound for the treatment of individuals.

Toxicity and therapeutic efficacy of modified netrin-1 peptides according to the instant invention and compositions thereof can be determined using cell cultures and/or experimental animals and pharmaceutical procedures in the art. For example, one may determine the lethal dose, LC₅₀ (the dose expressed as concentration x exposure time that is lethal to 50% of the population) or the LD₅₀ (the dose lethal to 50% of the population), and the ED₅₀ (the dose therapeutically effective in 50% of the population) by methods in the art. The dose ratio between toxic and therapeutic effects is the therapeutic index and it can be expressed as the ratio LD₅₀/ED₅₀. Modified netrin-1 peptides which exhibit large therapeutic indices are preferred. While modified netrin-1 peptides that result in toxic side-effects may be used, care should be taken to design a delivery system that targets such compounds to the site of treatment to minimize potential damage to uninfected cells and, thereby, reduce side-effects.

The data obtained from the cell culture assays and animal studies can be used in formulating a range of dosages for use in humans. Preferred dosages provide a range of circulating concentrations that include the ED₅₀ with little or no toxicity. The dosage may vary depending upon the dosage form employed and the route of administration utilized. Therapeutically effective amounts and dosages of one or more modified netrin-1 peptides according to the present invention can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography. Additionally, a dosage suitable for a given subject can be determined by an attending physician or qualified medical practitioner, based on various clinical factors.

The following examples are intended to illustrate but not to limit the invention.

### Modified Netrin-1 Peptides Induce Unexpectedly Superior Cardioprotection Against Ischemia/Reperfusion Injury

Netrin-1 and the prior art peptides V1, V2, and V3 are cardioprotective via DCC/ERK1/2/eNOSₛ₁₁₇₇/NO signaling in the heart. When administered before an ischemia/reperfusion injury, V1, V2, or V3 perfused hearts had a substantial reduction in infarct size (Control I/R: 39.3±0.3% vs. I/R+V1: 14.6±2.3% vs. I/R+V2: 23.0±2.8% vs. I/R+V3: 18.8±0.8%, p<0.001). Thus, the V1, V2, and V3 prior art peptides are highly effective in inducing cardioprotection against ischemia/reperfusion injuries. Furthermore, when administered after an ischemia/reperfusion injury, all three prior art peptides also induced potent cardioprotection against I/R injury (Control I/R: 37.6±1.3% vs. I/R+V1: 17.6±3.2% vs. I/R+V2: 20.6±1.7% vs. I/R+V3: 15.8±2.0%, p<0.001).

Fragments of the prior art peptides, e.g., V1-9aa, V2-10aa, and V3-11aa, also significantly reduced the infarct size compared to control group (Control I/R: 37.6±1.3% vs. I/R+V1-9aa: 16.8±2.2%; I/R+V2-10aa: 18.6±1.7%; I/R+V3-11aa: 16.7±3.0%, p<0.001). See Table 1:

TABLE 1

| Prior Art | Infarct Size | Infarct Size | % MI | |
|---|---|---|---|---|
| Peptides | Mean (%) | SEM (%) | Reduction | N |
| I/R Control | 37.6 | 1.3 | | 5 |
| I/R + V1-9aa | 16.8 | 2.2 | 55% | 3 |
| I/R + V2-10aa | 18.6 | 1.7 | 51% | 3 |
| I/R + V3-11aa | 16.7 | 3.0 | 56% | 4 |

Thus, ability of modified netrin-1 peptides to reduce infarct size was similarly measured and the results are summarized in Table 2:

TABLE 2

| Modified Netrin-1 | Infarct Size | Infarct Size | % MI | |
|---|---|---|---|---|
| Peptides | Mean (%) | SEM (%) | Reduction | N |
| I/R Control | 43.1625 | 1.28995 | | 4 |
| I/R + V1P | 10.0100 | 0.72350 | 77% | 4 |
| I/R + V2P | 18.3315 | 1.65828 | 58% | 4 |
| I/R + V3P | 12.9700 | 2.42086 | 70% | 4 |
| I/R + V1S | 12.7750 | 1.92164 | 71% | 4 |
| I/R + V1T | 15.4650 | 2.07091 | 64% | 4 |
| I/R + V1C | 11.2700 | 0.81395 | 74% | 4 |

***p < 0.001 vs. Control

A comparison between the percent reduction in infarct size conferred by the modified netrin-1 peptides and the percent reduction in infarct size conferred by the closest prior art peptide evidences that the activities of the modified netrin-1 peptides are significantly superior to the prior art peptides as shown Table 3:

TABLE 3

| | % Improvement in |
|---|---|
| Comparison | MI Reduction |
| V1P vs. V1-9aa | (77 − 55)/55 = 40% |
| V2P vs. V2-10aa | (58 − 51)/51 = 14% |
| V3P vs. V3-11aa | (70 − 58)/70 = 25% |
| V1S vs. V1-9aa | (71 − 55)/55 = 29% |
| V1T vs. V1-9aa | (64 − 55)/55 = 16% |
| V1C vs. V1-9aa | (74 − 55)/55 = 35% |

Therefore, in some embodiments, the present invention provides one or more modified netrin-1 peptides which may be used to treat acute myocardial infarction in subjects. In some embodiments, the present invention is directed to treating myocardial infarction, reducing, or inhibiting infarct size, and/or reducing or inhibiting FR injury in a subject which comprises administering a therapeutically effective amount of one or more one or more modified netrin-1 peptides to the subject, before, during, or after the myocardial infarction or the ischemia or reperfusion.

The modified netrin-1 peptides are expected to exhibit robust inhibitory effects on post endothelial injury neointimal formation which simulates clinical restenosis post PCTA in a manner similar to the full-length netrin-1 peptide. Additionally, the modified netrin-1 peptides are expected to attenuate restenosis via augmentation of endothelial progenitor cell (EPC) function to enhance re-endothelialization at the wound area (e.g., endothelial cell damage of the coronary arteries caused by the angioplasty procedure), and increased nitric oxide production to inhibit vascular smooth muscle cell (VSMC) proliferation and migration. Therefore, in some embodiments, the present invention is directed to treating or inhibiting restenosis in a subject which comprises administering to the subject a therapeutically effective amount of one or more one or more modified netrin-1 peptides, before, during, or after an injury to endothelial cells in a blood vessel.

### Peptide Sequences

The following are the amino acid sequences of the modified netrin-1 peptides exemplified herein:

V1P: (mini-PEG)-CDCRHNTAG (SEQ ID NO: 2)
V2P: (mini-PEG)-CLNCRHNTAG (SEQ ID NO: 3)
V3P: (mini-PEG)-CPCKDGVTIGIT (SEQ ID NO: 4)

### V1S: SDCRHNTAG (SEQ ID NO: 5)

### V1T: TDCRHNTAG (SEQ ID NO: δ)

V1C: CDCRHNTAG (SEQ ID NO: 7), wherein the cysteine residues are joined by a disulfide bond

The following are the amino acid sequences of the prior art peptides exemplified herein:

V1:
(SEQ ID NO: 8)
CKCNGHAARCVRDRDDSLVCDCRHNTAGPECDRCKPFHYDRPWQRATAR
EANEC
V2:
(SEQ ID NO: 9)
CNCNLHARRCRFNMELYKLSGRKSGGVCLNCRHNTAGRHCHYCKEGYYR
DMGKPITHRKAC
V3:
(SEQ ID NO: 10)
CDCHPVGAAGKTCNQTTGQCPCKDGVTGITCNRCAKGYQQSRSPIAPC
V1-9 aa:
(SEQ ID NO: 11)
CDCRHNTAG
V2-10 aa:
(SEQ ID NO: 12)
CLNCRHNTAG
V3-11 aa:
(SEQ ID NO: 13)
CPCKDGVTGIT
Negative control V3-16 aa:
(SEQ ID NO: 14)
HPVGAAGKTCNQTTGQ

The accession number of the full-length netrin-1 human sequence is GI 148613884.

### Materials and Methods

Various methods disclosed in PCT/US2011/038277 and PCT/US2015/023248 may be employed. Because the experiments herein show that the exemplified modified netrin-1 peptides exhibit superior activity the prior art peptides, modified netrin-1 peptides may be used in place of or to supplement therapeutic treatments employing netrin-1 or other netrin-1 derivatives such as the prior art peptides.

### Materials

Purified mouse netrin-1 was purchased from R&D Systems (Minneapolis, Minn., USA). Peptide fragment V1 (285-338 amino acid of human netrin-1), V2 (341-401 aa), V3 (404-451 aa), V1-9aa (304-312 aa), V2-10aa (368-377 aa), V3-16aa (407-422 aa), and V3-11aa (423-433 aa) were synthesized by GenicBio Limited (Shanghai, CHN). Polyclonal antibodies specific for phosphorylated ERK1/2, ERK1/2, and eNOSₛ₁₁₇₉ were obtained from Cell Signaling Technology (Danvers, Mass., USA). Monoclonal antibody for eNOS was purchased from BD Biosciences (San Jose, Calif., USA).

### Cell Culture

Bovine aortic endothelial cells (BAECs, Cell Systems, Kirkland, Wash., USA) were cultured in media 199 containing 10% fetal bovine serum (FBS) as previously described. See Chalupsky & Cai, PNAS USA 2005; 102:9056-9061; and Nguyen & Cai, PNAS USA 2006; 103:6530-6535. One day post confluent cells were starved in media containing 5% FBS overnight, then stimulated with netrin-1 protein or different peptide fragments, and harvested at different time points.

### Western Blotting

For Western blotting, approximately 20-40 μg of protein was separated by 10% SDS-PAGE, transferred to nitrocellulose membranes, and probed with phosphorylated ERK1/2, ERK1/2, eNOS, and eNOSₛ₁₁₇₉ (1:1,000) antibodies using methods known in the art. See e.g., Gao et al., Journal of Molecular and Cellular Cardiology. 2009; 47:752-760.

### Langendorff Perfusion

Male C57BL/6J mice (8-12 weeks old) were obtained from the Charles River Laboratories (Wilmington, Mass., USA). After anesthetized with intraperitoneal pentobarbitone (60 mg/kg), mouse hearts were harvested immediately and the aortas were cannulated with a 20-gauge stainless steel blunt needle and transferred to the Langendorff rig and perfused retrograde instantly with modified Krebs-Henseleit buffer (KHB) for 30 minutes as previously described. See Bouhidel et al. Front Biosci (Landmark Ed) 2014; 19:566-570, and Zhang et al., J Mol Cell Cardiol. 2010; 48:1060-1070. Then hearts were pre-perfused for 45 minutes with or without netrin-1 (100 ng/ml), or different peptide fragments at the same molar concentration 1.47 nmol/L as netrin-1, prior to being subjected to ischemia/reperfusion (I/R) injury (a 20-minute global ischemia followed by a 60-minute reperfusion with or without netrin-1 or different peptide fragments). Hearts were then harvested for analyses of infarct size. For post-conditioning treatment with peptide fragments, hearts underwent 40 minutes of KHB perfusion, 20 minutes of global ischemia, and a 60-minute reperfusion with different peptide fragments.

### Infarct Size Analysis

At the end of I/R protocol, hearts were sliced perpendicular to the long-axis of the heart at 1 mm intervals and stained with 1% triphenyl tetrazolium chloride (TTC) in PBS for 10 minutes at room temperature. After washing with PBS once, sections of the hearts will be fixed in 10% formalin overnight. The heart slices were then digitally photographed for planimetry using NIH Image 1.62. Infarct size is expressed as an infarct-to-risk zone ratio (the risk zone is the whole ventricular volume in this global ischemic model).

### Statistical Analysis

Densitometric data of western blotting was obtained by software Image J. Grouped data was analyzed by software Gradpad Prism δ. All values are expressed as Mean±SEM. Comparisons of more than two groups were performed using a one way ANOVA analysis with Newman-Keuls test as a post-hoc test. Statistical significance is set as p<0.05.

All scientific and technical terms used in this application have meanings commonly used in the art unless otherwise specified.

As used herein, the terms “subject”, “patient”, and “individual” are used interchangeably to refer to humans and non-human animals. The term “non-human animal” includes all vertebrates, e.g., mammals and non-mammals, such as non-human primates, horses, sheep, dogs, cows, pigs, chickens, and other veterinary subjects and test animals. In some embodiments of the present invention, the subject is a mammal. In some embodiments of the present invention, the subject is a human.

As used herein, the term “diagnosing” refers to the physical and active step of informing, i.e., communicating verbally or by writing (on, e.g., paper or electronic media), another party, e.g., a patient, of the diagnosis. Similarly, “providing a prognosis” refers to the physical and active step of informing, i.e., communicating verbally or by writing (on, e.g., paper or electronic media), another party, e.g., a patient, of the prognosis.

The use of the singular can include the plural unless specifically stated otherwise. As used in the specification and the appended claims, the singular forms “α”, “an”, and “the” can include plural referents unless the context clearly dictates otherwise.

As used herein, “and/or” means “and” or “or”. For example, “A and/or B” means “A, B, or both A and B” and “A, B, C, and/or D” means “A, B, C, D, or a combination thereof” and said “A, B, C, D, or a combination thereof” means any subset of A, B, C, and D, for example, a single member subset (e.g., A or B or C or D), a two-member subset (e.g., A and B; A and C; etc.), or a three-member subset (e.g., A, B, and C; or A, B, and D; etc.), or all four members (e.g., A, B, C, and D).

As used herein, the phrase “one or more of”, e.g., “one or more of A, B, and/or C” means “one or more of A”, “one or more of B”, “one or more of C”, “one or more of A and one or more of B”, “one or more of B and one or more of C”, “one or more of A and one or more of C” and “one or more of A, one or more of B, and one or more of C”.

The phrase “comprises, consists essentially of, or consists of A” is used as a tool to avoid excess page and translation fees and means that in some embodiments the given thing at issue: comprises A, consists essentially of A, or consists of A. For example, the sentence “In some embodiments, the composition comprises, consists essentially of, or consists of A” is to be interpreted as if written as the following three separate sentences: “In some embodiments, the composition comprises A. In some embodiments, the composition consists essentially of A. In some embodiments, the composition consists of A.”

Similarly, a sentence reciting a string of alternates is to be interpreted as if a string of sentences were provided such that each given alternate was provided in a sentence by itself. For example, the sentence “In some embodiments, the composition comprises A, B, or C” is to be interpreted as if written as the following three separate sentences: “In some embodiments, the composition comprises A. In some embodiments, the composition comprises B. In some embodiments, the composition comprises C.” As another example, the sentence “In some embodiments, the composition comprises at least A, B, or C” is to be interpreted as if written as the following three separate sentences: “In some embodiments, the composition comprises at least A. In some embodiments, the composition comprises at least B. In some embodiments, the composition comprises at least C.”

As used herein, the terms “protein”, “polypeptide”, “peptide”, and “peptide fragments” are used interchangeably to refer to two or more natural and/or unnatural amino acids linked together and one letter amino acid designations are used in the sequences and formulas herein. As used herein, “aa” is an abbreviation used for “amino acids”. For example, the “9aa” of “V1-9aa” indicates that the peptide is 9 amino acid residues long.

To the extent necessary to understand or complete the disclosure of the present invention, all publications, patents, and patent applications mentioned herein are expressly incorporated by reference therein to the same extent as though each were individually so incorporated.

Having thus described exemplary embodiments of the present invention, it should be noted by those skilled in the art that the within disclosures are exemplary only and that various other alternatives, adaptations, and modifications may be made within the scope of the present invention. Accordingly, the present invention is not limited to the specific embodiments as illustrated herein, but is only limited by the following claims.

1. A peptide comprising, consisting essentially of, or consisting of SEQ ID NO: 1 as follows:
(SEQ ID NO: 1)
X1-X2-X3-C-X4-X5-X6-X7-T-X8-G
wherein
X1 is Ala, Asn, Cys, Ser, or Thr, preferably X1 is Cys, Ser, or Thr, wherein when X1 is Cys, it is attached, covalently or non-covalently, to either the cysteine residue at the fourth amino acid position via a disulfide bond or an ethylene oxide compound;
X2 is present or absent, and if present, X2 is Ala, Ile, Leu, Met, Phe, Pro, Trp, or Val, preferably X2 is Leu or Pro;
X3 is present or absent, and if present, X3 is Asn, Asp, Cys, Gln, Glu, Gly, Ser, Thr, or Tyr, preferably X3 is Asn or Asp;
X4 is Arg, His, or Lys, preferably X4 is Arg or Lys;
X5 is Arg, Asp, Glu, His, Lys, Phe, Trp, or Tyr, preferably X5 is Asp or His;
X6 is Asn, Cys, Gln, Gly, Ser, Thr, or Tyr, preferably X6 is Asn or Gly;
X7 is present or absent, and if present, X7 is Ile, Thr, or Val, preferably X7 is Val; and
X8 is present or absent, and if present, X8 is Ala, Ile, Leu, Met, Phe, Pro, Trp, or Val, preferably X8 is Ala; and
wherein X2, X3, or both X2 and X3 are present.

2. The peptide according to claim 1, wherein the ethylene oxide compound is polyethylene glycol (PEG), polyethylene oxide (PEO), and polyoxyethylene (POE), methoxypolyethylene glycol (MPEG), or monomethoxypolyethylene glycol (mPEG), preferably the ethylene oxide compound is mini-PEG.

3. The peptide according to claim 1, wherein the peptide comprises, consists essentially of, or consists of SEQ ID NO: 2, SEQ ID NO: 3, SEQ ID NO: 4, SEQ ID NO: 5, SEQ ID NO: δ, or SEQ ID NO: 7.

4. The peptide according to claim 1, wherein the peptide is about 8-60, about 8-55, about 8-50, about 8-45, about 8-40, about 8-35, about 8-30, about 8-25, about 8-20, about 8-15, about 8-12, about 8-11, about 9-60, about 9-55, about 9-50, about 9-45, about 9-40, about 9-35, about 9-30, about 9-25, about 9-20, about 9-15, about 9-12, or 9-11 amino acid residues long.

5. The peptide according to claim 1, wherein the peptide is 8, 9, 10, or 11 amino acid residues long.

6. A composition comprising one or more peptides according to claim 1.

7. A method of stimulating, increasing, or enhancing nitric oxide production by endothelial cells, which comprises administering to the endothelial cells one or more peptides according to claim 1 or a composition thereof.

8. A method of stimulating or inducing phosphorylation of ERK1/2 and/or eNOS in endothelial cells, which comprises administering to the endothelial cells one or more peptides according to claim 1 or a composition thereof.

9. A method of treating, inhibiting, or reducing an injury to a tissue or organ having endothelial cells, said endothelial cells are vascular endothelial cells, which comprises stimulating, increasing, or enhancing nitric oxide production by the endothelial cells and/or stimulating or inducing phosphorylation of ERK1/2, eNOS, or both in the endothelial cells by administering to the endothelial cells, before, during, and/or after the injury, one or more peptides according to claim 1 or a composition thereof.

10. The method according to claim 7, wherein the administration to the endothelial cells is in vivo administration.

11. The method according to claim 10, wherein the injury is caused by superoxide production, ischemia/reperfusion, or myocardial infarction.

12. The method of claim 11, wherein the tissue is cardiac tissue or the organ is a heart.

13. The method of claim 12, wherein the injury is caused by myocardial infarction and the administration reduces the infarct size of the heart.

14. A method of treating, inhibiting, or reducing an ischemia/reperfusion injury to a heart in a subject, comprising administering a therapeutically effective amount of one or more peptides according to claim 1 or a composition thereof to the subject, thereby treating, inhibiting, or reducing the ischemia/reperfusion injury.

15. A method of decreasing or reducing the infarct size of a heart in a subject resulting from an ischemia/reperfusion injury, comprising administering a therapeutically effective amount of one or more peptides according to claim 1 or a composition thereof to the subject, thereby decreasing or reducing the infarct size.

16. The method according to claim 8, wherein the administration to the endothelial cells is in vivo administration.

17. The method according to claim 9, wherein the administration to the endothelial cells is in vivo administration.

18. The method according to claim 14, wherein the administration to the endothelial cells is in vivo administration.

19. The method according to claim 15, wherein the administration to the endothelial cells is in vivo administration.
